SEQUENCE LISTING (26 sequences)

SEQ ID NO: 1 - DNA, 22 bp, Artificial
  Staphylococcus aureus mecA gene forward primer
tggtatgtggaagttagattgg

SEQ ID NO: 2 - DNA, 22 bp, Artificial
  Staphylococcus aureus mecA gene reverse primer
ggatctgtactgggttaatcag

SEQ ID NO: 3 - DNA, 23 bp, Artificial
  Staphylococcus aureus nuc gene forward primer
atagggatggctatcagtaatgt

SEQ ID NO: 4 - DNA, 22 bp, Artificial
  Staphylococcus aureus nuc gene reverse primer
gacctgaatcagcgttgtcttc

SEQ ID NO: 5 - DNA, 21 bp, Artificial
  Staphylococcus aureus 16S rRNA gene forward primer
tagccgacctgagagggtgat

SEQ ID NO: 6 - DNA, 21 bp, Artificial
  Staphylococcus aureus 16S rRNA gene reverse primer
gtagttagccgtggctttctg

SEQ ID NO: 7 - DNA, 22 bp, Artificial
  Staphylococcus aureus agr gene left primer
gccataaggatgtgaatgtatg

SEQ ID NO: 8 - DNA, 22 bp, Artificial
  Staphylococcus aureus agr gene right primer
cagctatacagtgcatttgcta

SEQ ID NO: 9 - DNA, 22 bp, Artificial
  Staphylococcus aureus clumping factor gene left primer
ggctactggcataggtagtaca

SEQ ID NO: 10 - DNA, 22 bp, Artificial
  Staphylococcus aureus clumping factor gene right primer
gctgaatctgaaccactatctg

SEQ ID NO: 11 - DNA, 22 bp, Artificial
  Staphylococcus aureus 16S rRNA gene left primer
ggattagataccctggtagtcc

SEQ ID NO: 12 - DNA, 21 bp, Artificial
  Staphylococcus aureus 16S rRNA gene right primer
cttcgggtgttacaaactctc

SEQ ID NO: 13 - DNA, 21 bp, Artificial
  Staphylococcus aureus hld gene left primer
attagggatgcaggtcttagc

SEQ ID NO: 14 - DNA, 22 bp, Artificial
  Staphylococcus aureus hld gene right primer
ctataagctgcgatgttaccaa

SEQ ID NO: 15 - DNA, 20 bp, Artificial
  Staphylococcus aureus femA gene left primer
taccgctttaaacgtggatt

SEQ ID NO: 16 - DNA, 22 bp, Artificial
  Staphylococcus aureus femA gene right primer
gatatcacacacttgcaaacac

SEQ ID NO: 17 - DNA, 21 bp, Artificial
  Staphylococcus aureus rho terminaton factor gene left primer
aacaatctggtttaggtcgtg

SEQ ID NO: 18 - DNA, 22 bp, Artificial
  Staphylococcus aureus rho termination factor gene right primer
tggaatgattcatactgaggag

SEQ ID NO: 19 - DNA, 22 bp, Artificial
  Staphylococcus aureus DNA polymerase III gene left primer
gtagaattaacgcaacatcacc

SEQ ID NO: 20 - DNA, 22 bp, Artificial
  Staphylococcus aureus DNA polymerase III gene right primer
cacgctgtacctaccaataatc

SEQ ID NO: 21 - DNA, 22 bp, Artificial
  Staphylococcus aureus nuclease (nuc) gene left primer
gtcctgaagcaagtgcatttac

SEQ ID NO: 22 - DNA, 22 bp, Artificial
  Staphylococcus aureus nuclease (nuc) gene right primer
gacctgaatcagcgttgtcttc

SEQ ID NO: 23 - DNA, 22 bp, Artificial
  Staphylococcus aureus 23S rRNA gene left primer
atttgagaggagctgtccttag

SEQ ID NO: 24 - DNA, 22 bp, Artificial
  Staphylococcus aureus 23S rRNA gene right primer
attagtattcgtcagctccaca

SEQ ID NO: 25 - DNA, 22 bp, Artificial
  Staphylococcus aureus heat shock protein 60 kDA (hsp60) gene left primer
gacaaagcagttaaagttgctg

SEQ ID NO: 26 - DNA, 22 bp, Artificial
  Staphylococcus aureus heat shock protein 60 kDA (hsp60) gene right primer
ccttcaaccacttctagttcag

(19) United States
(12) Patent Application Publication
(10) Pub. No.: US 2004/0110138 A1
(43) Pub. Date: Jun. 10, 2004

(54) Method for the detection of multiple genetic targets

(75) Inventors: Paul Lem, Scarborough (CA); Jamie Spiegelman, Toronto (CA)

Correspondence Address: GREENBERG TRAURIG, LLP, ONE INTERNATIONAL PLACE, 20th FL, ATTN: PATENT ADMINISTRATOR, BOSTON, MA, 02110, US

(73) Assignee: University of Ottawa, Ottawa (CA)

(21) Appl. No.: 10/315,217

(22) Filed: Dec. 10, 2002

(51) Int. Cl. C12Q 1/68; C12P 19/34

(52) U.S. Cl. 435/6; 435/91.2

(57) ABSTRACT

A method for simultaneous amplification and detection of multiple genetic targets is provided. Furthermore, a primer design protocol specific to the PCR reaction conditions of the present invention is also provided. The method of the present invention includes a PCR reaction mixture and primers specifically selected according to the reactions conditions provided. Multiple genetic targets are amplified simultaneously by this method, without requiring optimization of the reaction conditions.

### CROSS-REFERENCE TO RELATED APPLICATIONS

This application claims priority to U.S. Provisional Application Serial No. 60/______, filed Nov. 1, 2002, the text of which is expressly incorporated herein by reference.

### TECHNICAL FIELD

This invention relates to a method for the amplification and detection of multiple genetic targets, and components thereof. More specifically, the present invention relates to the simultaneous amplification of multiple genetic targets in a single reaction. In particular, the present invention relates to an enhanced Polymerase Chain Reaction (PCR) method and components thereof.

### BACKGROUND OF THE INVENTION

The Polymerase Chain Reaction (PCR) is a widely used technique that employs a thermostable DNA polymerase enzyme in conjunction with target-specific primers to amplify target genetic sequences. The term “multiplex” refers to the ability to amplify multiple genetic sequences simultaneously, in a single reaction vessel, rather than having to conduct each amplification reaction individually. Thus, saving time and resources. Multiplex PCR generally refers to a multiplex reaction using standard PCR reagents and unmodified deoxynucleotide triphosphates (dNTPs) for amplifying multiple genetic sequences simultaneously. However, limitations exist in the number of target sequences that are efficiently amplified with current multiplex PCR systems. The products of such a multiplex PCR reaction are routinely detected by a simple technique like agarose gel electrophoresis and staining by a common dye like ethidium-bromide. Other technologies “multiplex” multiple genetic targets at one time, but then use detection methods other than agarose gel electrophoresis. For example, the Roche Light Cycler™ can multiplex more than one genetic target at a time, but detects amplified products using hybridization probes employing Fluorescent Resonance Energy Transfer (FRET) technology. Technologies like the GenePrint PowerPlexw™ system incorporate fluorescently-labeled nucleotides into the PCR products, for later fluorescent detection. Although these systems often enhance the detection capacity of multiplex PCR, they are technology-dependent, and expensive. There remains a need for a simple multiplex PCR system that in reliable, efficient and cost-effective.

Many researchers have designed diagnostic assays based on multiplex PCR principles. Most multiplex PCR protocols are limited to the successful amplification of just a few target genetic sequences in a single reaction. Several studies have been able to simultaneously amplify 3 to 5 multiplex PCR products (Maes et al., 2002; Paton and Paton, 2002). A study by Henegariu et al. (1997) reported visualization of 7 multiplex PCR products, but required extensive optimization of reaction reagents and Conditions in order to do so. Other studies use as many as seven primer pairs in the same reaction tube, but this is done with the expectation that the seven genetic targets will not all be present in the sample of interest (Markoulatos et al., 2001; Elsayed et al., 2001; Hindiyeh et al., 2001).

Furthermore, multiplex PCR reaction conditions need to be empirically optimized depending on the primers used (Henegariu et al., 1997). Even then, there is no certainty that a given primer pair will work with others in a multiplex PCR assay. Also, the addition or removal of a primer pair in an existing working assay may require the optimization process to begin all over again. It would be useful to have a single set of multiplex PCR reaction conditions that do not require optimization each time a different primer is added or removed from the assay.

U.S. Pat. Nos. 5,882,856 and 6,207,3721 relate to a universal primer sequence for multiplex DNA amplification. In particular, these patents disclose chimeric primers that serve as high stringency recognition sequences in the amplification process and normalize the degree of amplification of different targets. Although these primers claim to have a uniformly high degree of specificity on the annealing reactions that occur between different primers present in the mixture and their cognate target sequences in the DNA template without requiring the need to adjust multiplex reaction conditions, the design of these primers is both complex and time consuming. Clearly, a technician could not readily optimize these primers for use in a DNA amplification protocol, nor replace a given set of primers in the midst of an amplification assay.

U.S. Pat. No. 6,333,179 relates to methods and compositions for multiplex amplification of nucleic acids. According to this patent, a predetermined ratio of primers can be calculated according to a disclosed formula to achieve approximate equi-molar yield of multiplex PCR products. According to this formula, primer concentrations are varied as a function of amplicon length. Such a method is time-consuming, requiring individual calculations for each primer pair. It would desirable to have a method for simultaneously amplifying multiple genetic targets that could be repeatedly performed according to a set of common directions without requiring optimization of the reaction conditions.

In many cases it is desirable to be able to simultaneously amplify numerous genetic targets in a convenient and cost-effective manner. In the field of infectious disease, for example, often a practitioner is interested in pinpointing a causative agent of infection from a large group of potential organisms. It would be beneficial to have a multiplex PCR system capable of simultaneously amplifying more than 5 target genetic sequences without the need for optimization steps. Thereby providing the capability to quickly and conveniently detect a genetic target of interest.

In summary, there is a need for the ability to efficiently and economically amplify and detect multiple genetic targets in a single reaction vessel, without the need for optimization of multiplex PCR reaction conditions.

In particular, in the case of genetic screening for diseases there is truly a need for a method for simultaneously amplifying multiple genetic targets without requiring optimization of reaction conditions.

### SUMMARY OF THE INVENTION

An enhanced multiplex PCR method for simultaneous amplification of multiple genetic targets is provided. According to the present invention, multiple genetic targets can be quickly and easily detected without requiring extensive optimization of the enhanced multiplex PCR method herein described. Furthermore, a primer design protocol specific to the PCR reaction conditions of the present invention is also provided. Amplification of at least ten genetic targets simultaneously in single reaction vessel is provided in accordance with an embodiment of the present invention. In addition, the method of the present invention is pre-optimized for amplification of multiple genetic targets and can be performed with primers designed according to a design protocol without the need for optimization of the multiplex PCR reaction conditions.

It is an object of the present invention to provide an enhanced multiplex PCR method for simultaneously amplifying multiple genetic targets in a single reaction vessel.

It is a further object of the present invention to provide an enhanced multiplex PCR method for simultaneously amplifying multiple genetic targets in a single reaction vessel without requiring optimization of the reaction conditions.

It is another object of the present invention to provide a novel PCR reaction mixture adaptable for simultaneously amplifying multiple genetic targets in a single reaction vessel.

It is a further object of the present invention to provide primer pairs designed for use in an enhanced multiplex PCR method.

It is yet a further object of the present invention to provide a kit adaptable for simultaneously amplifying multiple genetic targets in a single reaction vessel.

### BRIEF DESCRIPTION OF THE DRAWINGS

Further features and advantages of the present invention will become apparent from the following detailed description, taken in combination with the appended drawings, in which:

FIG. 1 is a flowchart exemplifying an enhanced multiplex PCR method in accordance with an aspect of the present invention;

FIG. 2 illustrates a primer selection criteria in accordance with an aspect of the present invention;

FIG. 3 is a flowchart of a series of PCR Cycling steps performed in accordance with an aspect of the present invention;

FIG. 4 illustrates the detection of ten different genes in methicillin-resistant Staphylococcus aureus in accordance with an aspect of the present invention;

FIG. 5 illustrates the detection sensitivity of ten different genetic targets of the present invention as displayed at varying initial concentrations of bacteria in accordance with an aspect of the present invention;

FIG. 6 illustrates the effect of varying magnesium chloride concentrations on the detection of ten different genetic targets in accordance with an aspect of the present invention; and

FIG. 7 illustrates the effects of varying primer concentrations on the detection of ten different genetic targets in accordance with an aspect of the present invention.

### DETAILED DESCRIPTION OF THE PREFERRED EMBODIMENT

An enhanced multiplex Polymerase Chain Reaction (PCR) method for reliable and efficient amplification of multiple genetic targets in a single reaction vessel is provided by the present invention. The ability to simultaneously amplify and detect multiple genetic targets using a convenient and time-efficient method of enhanced multiplex PCR as herein provided, provides a clear advantage over the prior art. The present invention provides a simple, efficient and economical method for achieving multiple target genetic sequence amplification in a single reaction. In particular, products of the enhanced multiplex PCR method can be detected by routines and cost effective methods, such as agarose gel electrophoresis, for example.

“Amplification” of DNA as used herein denotes the use of polymerase chain reaction (PCR) to increase the concentration of a particular DNA sequence within a mixture of DNA sequences.

An “amplicon” is a product of the amplification of a target genetic sequence.

“Multiplex PCR” as used herein refers to the use of the polymerase chain reaction (PCR) for simultaneous amplification of multiple genetic targets in a single polymerase chain reaction (PCR) reaction. PCR as used herein may include touch-down PCR.

A “PCR reaction mixture” as used herein denotes a mixture adaptable for simultaneously amplifying multiple genetic targets under suitable conditions for PCR,

A “genetic target” as used herein denotes a genetic sequence capable of amplification by polymerase chain reaction (PCR). A genetic target in accordance with the present invention includes any DNA sequence, including bacterial, viral, human, plant, and animal DNA, for example.

It is technically more challenging to design a multiplex PCR system that can actually amplify multiple genetic targets. Different primer pairs have different amplification efficiencies, thus making it difficult to achieve adequate amplification of all primer pairs simultaneously. Due to the iterative nature of PCR cycles, amplicons generated by more efficient primer pairs quickly become the dominant species in the mix. This dominant species then out-competes the less efficient primer pairs for PCR reagents, resulting in insufficient amplification of the less efficient species.

The present invention provides an enhanced multiplex PCR reaction method that includes pre-selected primer pairs for use in amplifying genetic targets. The enhanced multiplex PCR reaction method of the present invention further includes pre-set amplification reaction conditions. Pre-selected primer pairs of the present invention are target specific primer pairs designed according to a given primer design protocol. According to one embodiment of the present invention, when employed under pre-set amplification reaction conditions, a predetermined concentration of the primer pairs optimize the amplification efficiency of the genetic targets of interest. The unique reaction conditions of the present invention further serve to optimize the amplification efficiencies of the target specific primer pairs to effectively amplify multiple genetic targets simultaneously. According to a preferred embodiment of the invention 10 or more genetic targets can be simultaneously amplified in the same reaction vessel. The present invention can be employed with standard PCR equipment while achieving excellent sensitivity and specificity for the detection of multiple genetic targets.

Unique reaction conditions coupled with target-specific primer pairs and a series of thermal cycling conditions provide an enhanced multiplex PCR system that reliably and efficiently amplifies multiple target genetic sequences simultaneously, in accordance with one embodiment of the present invention. Primer pairs of the present invention are designed according to a primer design protocol or selected according to a primer selection criteria and employed in the enhanced multiplex PCR method in predetermined concentrations. The predetermined concentration of each primer pair in the reaction mixture of the present invention is preferably the same. According to one embodiment of the present invention, the predetermined concentration of the primer pairs in the reaction mixture does not interfere with the pre-set optimization reaction conditions of the enhanced multiplex PCR method. Furthermore, target-specific primer pairs of the present invention can be added, removed or replaced in the reaction mixture of the present invention without requiring re-optimization of the reaction conditions. According to this embodiment, the concentration of the primer pairs are predetermined with respect to the reaction conditions and allow for efficient amplification of all genetic targets. As provided in accordance with the present invention, the competing efficiency of the primer pairs in the reaction mixture is reduced, and thus does not effect the optimization of the reaction conditions. This aspect of the invention is a significant improvement over the prior art. The present invention is easily tailored to amplify a preferred number of target genetic sequences without requiring time-consuming optimization steps. For example, in the event that a sample is scheduled to be screened for 9 genetic targets, and it is subsequently determined that an additional genetic target is also of interest, the present invention may be adapted to accommodate the screening of all 10 genetic, targets, simultaneously. In doing so, the primer design protocol or primer selection criteria of the present invention would be used to obtain suitable primer pairs for the additional genetic targets of interest. In accordance with the method of the present invention as herein disclosed, these additional primer pairs would be added to the reaction mixture to provide the predetermined concentration. Likewise, primer pairs can be removed from the reaction mixture of the present invention without disturbing the optimization conditions of the reaction. In accordance with another embodiment of the present invention primer pairs can be replaced by other primer pairs as designed or selected according to the primer design protocol or primer selection criteria respectively, as herein described.

In summary, the present invention provides the powerful new ability to test for multiple genetic targets simultaneously without the frustration of repeatedly optimizing reaction conditions. The present invention can be readily incorporated into existing PCR products;, or used to design a new generation of screening tests. In this manner a diagnostic screening assay of the present invention can be easily performed by a clinician and results rapidly obtained. It is fully contemplated that the present invention includes a kit providing the materials for performing the enhanced multiplex PCR method herein described. The present invention has particular application in the diagnosis of infectious diseases where many target organisms can be simultaneously screened in a timely and affordable fashion. The present invention has the potential to be applied to many other areas of DNA-based diagnostics.

### MATERIALS AND METHODS

### MATERIALS

Primer Design

Primers should be selected to have melting temperatures in the range of 55 to 65° C. For the purposes of the present invention the primers are preferably selected to have a melting temperature within an approximate 2° C. range. More preferably, the melting temperatures of the primers are between 55° C. to 57° C. Standardizing the melting temperature to a set range facilitates the uniformity of the primer hybridization kinetics.

Amplicon Length

The primers of the present invention may be designed to produce a PCR product or amplicon of virtually any size. In accordance with the present invention a PCR product or amplicon may include a target genetic sequence as amplified and detected in accordance herewith. Typically amplicons of the present invention will range in size from 200 to 2000 base pairs (bp) or nucleotides (nt). According to a preferred embodiment of the present invention, primers are designed to produce a PCR product or amplicon that is less than 900 base pairs (bp) or nucleotides (nt) in length. More preferably, amplicons will range in length form 212 to 823 bp. It is fully contemplated that the present invention is adaptable for the amplification of amplicons larger than 2000 bp. In accordance with this embodiment, consideration should be given to the type of enzyme employed in connection with the present invention as well as the means used for detecting the amplicon in question.

In accordance with another embodiment of the present invention, amplicons are preferably of different lengths. A 20 bp difference in amplicon length is preferred when detection of the amplicons includes agarose gel electrophoresis and ethidium bromide staining. Alternatively, a single base pair difference in amplicon length may also be detected in accordance with the present invention. In this instance, a detection system such as polyacrylamide gel electrophoresis may be employed. It should be understood that the present invention may be employed to amplify genetic targets producing amplicons of any size. Furthermore, the present invention may be employed with a variety of detection means.

GC Content

Primers may be designed to have a GC content ranges from 20 to 80%. According to a preferred embodiment of the present invention, primer GC content ranges from 40 to 50%.

PCR Reagents and Concentrations

Magnesium Chloride (MgCl₂)

In accordance with the present invention a PCR reaction mixture includes a 5 mM-12.5 mM final concentration of MgCl₂. Preferably, a final MgCl₂ concentration is between 5-10 mM. More preferably, a final concentration of 7.5 mM is provided.

Deoxynucleotidetriphosphates (dNTPs)

dNTP concentrations in the PCR reaction mixture of the present invention preferably ranges from 0.25 mM to 1.25 mM. More preferably, a final dNTP concentration of approximately 0.25 mM in a reaction mixture of the present invention is provided. In accordance with an embodiment of the present invention, a high concentration of dNTPs is provided to avoid the dNTP concentrations from becoming a limiting reagent in the reaction.

Enzyme

PCR enzymes known in the art may be employed in accordance with the present invention, such as Taq polymerase, for example. According to a preferred embodiment of the present invention, a hot start enzyme is employed, such as or Amplitaq Gold, for example. Alternatively, in the absence of a hot start enzyme, a manual hot start step may be employed together with a standard PCR enzyme, as known in the art. It is fully contemplated that other enzymes capable of amplifying genetic sequences may be employed in accordance with the present invention.

Primer Concentrations

Primers of the present invention are provided to have a final concentration of 0.005 μM to 0.05 μM. Preferably, the final primer concentrations of the present invention range from 0.005 μM to 0.01 μM. More preferably, the final primer concentration of the PCR react:ion mixture of the present invention is approximately 0.01 μM.

### METHODS

Enhanced Multiplex PCR

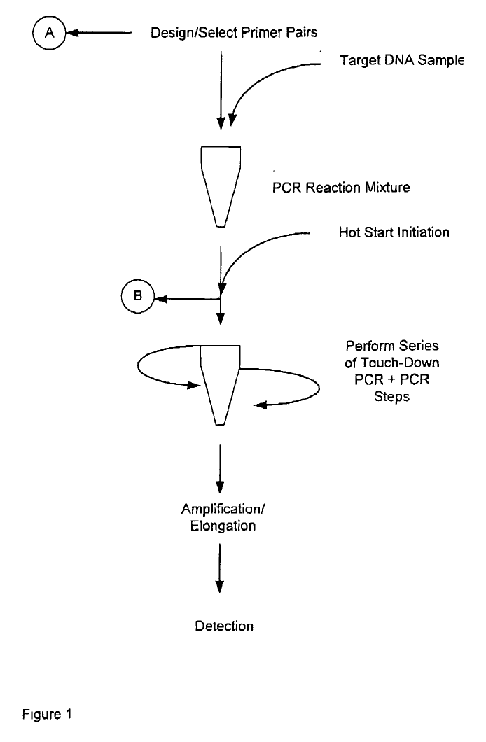

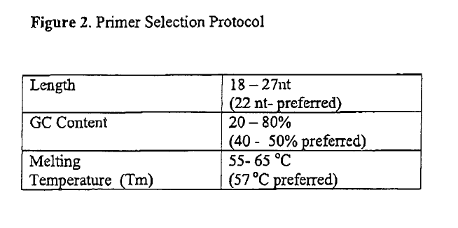

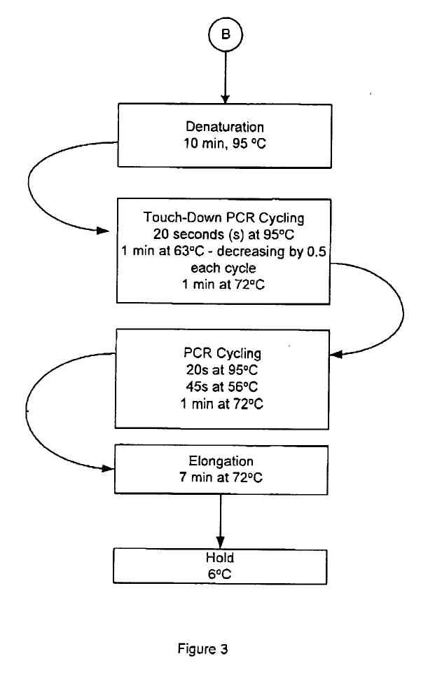

FIGS. 1-3 exemplify an enhanced multiplex PCR method according to a preferred embodiment of the present invention. FIG. 1 illustrates a step-wise method of the present invention. According to this embodiment of the invention, primer pairs are first selected or designed for specific genetic targets of interest in accordance with the criteria (A) outlined in FIG. 2. A reaction mixture is subsequently prepared and the primer pairs and a target DNA sample added thereto. A hot start initiation of amplification is subsequently initiated. A series of PCR steps are carried out, as exemplified in B (FIG. 3). Once amplification and elongation are complete, detection of the genetic targets is performed. According to the embodiment exemplified in FIG. 3 initial denaturation is conducted at 95° C. This denaturation step may be carried out for 5 to 10 minutes. Preferably, denaturation is carried out for 10 minutes.

Denaturation is followed by amplification. According to an embodiment of the invention a hot-start initiation of amplification is provided, as is known in the art. Preferably, amplification includes a series of PCR steps. PCR techniques applicable to the present invention include inter alia those described in PCR Primer: A Laboratory Manual, Dieffenback, C. W. and Dvekaler, G. S., eds., Cold Spring Harbor Laboratory Press (1995); Enzymatic amplification of beta-globin genomic sequences and restriction site analysis for diagnosis of sickle cell anemia, Saiki R K, Scharf S, Faloona F, Mullis K B, Horn G T, Erlich H A, Arnheim N, Science (1985) Dec. 20; 230(4732):1350-4. A first series of PCR steps preferably includes touchdown PCR. Touchdown or step-dowr. PCR refers to incremental decrease of the annealing temperature with each cycle. The objective is to increase the efficiency in each successive amplification step, while maintaining more rigorous primer specificity in the initial amplification steps. In accordance with a preferred embodiment of the present invention, 15 to 20 cycles of touchdown PCR are performed. More preferably, 20 cycles of touchdown PCR are performed.

Touchdown PCR may be followed by a second series of PCR steps. A second series of PCR steps preferably comprises of multiple cycles of regular PCR. A preferred range of regular PCR cycles is from 20 to 25 cycles. More preferably, 25 cycles of regular PCR are performed. It is fully contemplated that the PCR steps of the present invention may include steps of regular or touch-down PCR or a combination thereof.

A final elongation step may be performed following the PCR steps. Typically, elongation is performed at 72° C for approximately 7 minutes. Once elongation is complete, the reaction mixture may then be held at 6° C. prior to detection.

As indicated above the amplicons of the present invention may be detected according to any detection system or method known in the art.

The present invention will be more readily understood by referring to the following examples which are given to illustrate the invention rather than to limit its scope.

### EXAMPLE 1

In this study, we used a multiplex PCR assay that amplified the mecA, nuc, and 16S rRNA genes in bacterial DNA obtained directly from the blood culture bottle. This allowed us to identify S. aureus, determine methicillin resistance, and monitor successful amplification using the 16S rRNA gene as an internal control.

Blood culture bottles that were flagged as positive by the BacT/Alert system (Organon Teknika Corp., Durham N.C.) were processed by the Microbiology laboratory using standard microbiologic methods (Kloos & Bannerman, 1999). S. aureus was identified by a positive tube coagulase test. Oxacillin susceptibility for S. aureus was determined using the oxacillin salt agar screen method (NCCLS M100-S10, M7) and for coagulase-negative Staphylococci (CoNS) using disk diffusion testing (NCCLS M100-S10, M2).

DNA for PCR was prepared from blood culture bottle contents by the benzyl alcohol-guanidine hydrochloride organic extraction method according to Fredericks and Relman (1998). The PCR reaction mixture consisted of PCR Buffer (final concentration, 50 mM KCl, 9.0 mM MgCl₂, 10 mM Tris-HCl, pH 8.3), 0.25 mM each of dATP, dCTP, dTTP, and dGTP, 1.25 units of Taq DNA Polymerase (Roche Diagnostics), 2 μL of target DNA, and each of the 6 primers at 0.017 μM final concentration.

Primers were designed (using gene sequences obtained from GenBank) for mecA (accession # Y00688), nuc (accession # V01281 J01785 M10924), and 16S rRNA accession # AF076030). The primer sequences were as follows: mecA forward primer (SEQ ID NO. 1), 5′- TGGTATGTGGANGTTAGATTGG-3′ and reverse primer (SEQ ID NO. 2), 5′-GGATCTGTACTGGGTTAATCAG3′; nuc forward primer (SEQ ID NO. 3), 5′-ATAGGGATGGCTATCAGTAATGT-3′ and reverse primer (SEQ ID NO. 4), 5′-GACCTGAATCAGCGTTGTCTTC-3′; 16S rRNA forward primer (SEQ ID NO. 5) 5-′TAGCCGACCTGAGAGGGTGAT-3′ and reverse primer (SEQ ID NO. 6) 5′-GTAGTTAGCCGTGGCTTTCTG-3′. These primer pairs amplified a 1235 bp mecA fragment, 624 bp nuc fragment, and 228 bp 16S rRNA fragment, respectively.

PCR consisted of 40 cycles of amplification and was carried out in a GeneAmp PCR System 9600 thermal cycler. There was an initial heating step for 5 min at. 95° C. The first 20 cycles of touchdown PCR consisted of 20 s at 95° C., annealing for 45 s starting at 63° C. in the first cycle and decreasing by 0.5° C. for each of the subsequent 19 cycles, followed by extension for 45 s at 72° C. The last 20 cycles consisted of 20 s at 95° C., 45 s at 56° C., and 45 s at 72° C. Amplified products were detected by electrophoresis on 1.5% agarose gels that were stained with ethidium bromide and visualized under UV light.

In cases where there were discrepancies between the mecA PCR result of the blood culture bottle contents and the initial susceptibility result for the isolate recovered from the bottle, the isolate was re-tested for the mecA gene, oxacillin MIC was determined by agar dilution (NCCLS M100-S10, M7) and disk diffusion testing for oxacillin was repeated (NCCLS M100-S10, M2).

One hundred and twelve bottles were tested in this study. This consisted of 81 bottles that grew staphylococci, 19 bottles that grew other organisms, and 12 bottles that showed no growth of bacteria (Table 1). There were only 5 samples where the mecA PCR result of the bottle differed from the susceptibility result initially reported for the isolates recovered from the bottle. All 5 bottles contained CoNS and on retesting the isolates, the oxacillin disk diffusion and mecA PCR results agreed with the initial meca PCR result of the bottle (Table 2).

The direct PCR assay correctly identified all bottles with S. aureus and was more accurate than phenotypic testing for the determination of methicillin susceptibility of CoNS. In all 5 cases, where there was a discrepancy, repeat disk diffusion testing and mecA PCR of the isolates obtained results consistent with the initial PCR results.

The primary advantage of this multiplex assay is that it allows for the rapid identification of S. aureus and detection of methicillin resistance in Staphylococci in positive blood culture bottles growing Gram-positive cocci in clusters. PCR was performed on bacterial DNA obtained directly from the blood culture bottle, without the need for subculture. This assay takes approximately 4 h starting from the time a blood culture bottle is flagged positive to visualization of the PCR products on a gel. Conversely, it would require >24 h to obtain results using conventional phenotypic tests.

### EXAMPLE 2

Primer Design

Primers were designed to give amplicons that ranged in size between 200 to 900 bp. Where detection is by agarose gel electrophoresis, a size difference of at least 20 bp between individual amplicons is preferred. Detection by a more sensitive technique like capillary gel electrophoresis may be employed in accordance with the present invention to resolve a size difference of as little as 1 bp.

When designing primers, the structural properties of each primer set were selected according to the following primer selection criteria:

| | |
|---|---|
| Melting Temperature (Tₘ) | 55-57° C. |
| % GC | 40-50% |
| optimal primer length | 22 nt |
| Primer size (range) | 18-27 nt |

The above conditions were specified in a primer-design program such as Oligo4.0 or Primer3 (freely available at http://www-genome.wi.mit.edu/cgi-bin/primer/primer3_www.cgi), for example. After generating a possible candidate design, the new primer was checked for internal stability and mispriming according to methods well known in the art.

In cases where a primer pair did not work immediately, a new primer pair was designed according to the same principles, after reassessing the genomic structure and/or stability at that priming site. The reaction conditions were not adjusted in these cases.

PCR Reagents

| Reagent | Working Concentration |
|---|---|
| AmpliTaq Gold PCR Buffer | 10x |
| (500 mM KCl, 100 mM Tris HCl; | |
| pH 8.3) | |
| Magnesium chloride | 25 mM |
| dNTPs | 1.25 μM each |
| Enzyme | |
| AmpliTaq Gold | 5 U/μL |
| Primers | |
| Primer Master Mix | 0.05 μM each |
| (primers + TE Buffer: | |
| 10 mM Tris-HCl, pH 8.0 and 1 mM EDTA) | |

A hot start DNA polymerase enzyme was used in the PCR reaction steps, such as AmpliTaq Gold, for example. In this case, reactants were not wasted in the formation of unintended products and increased yields of the specific products were achieved. The final concentration of magnesium chloride in the reaction mix was 7.5 mM. Equal volumes of each primer were added together to make a mix containing all of the primers to be used in the multiplex assay. The concentration of each primer in this Primer Master Mix was 0.05 μM. As a result, the final primer concentration in the PCR reaction mixture was approximately 0.01 μM.

PCR Reaction Mixture

| Reagent | Volume (uL) | |
|---|---|---|
| Amplitaq Gold | 3 | |
| PCR Buffer | | |
| MgCl₂ | 9 | |
| dNTPS | 6 | |
| DNA template | 3 | |
| H₂0 | 3 | |
| AmpliTaq Gold | 0.5 | |
| Primer Master Mix | 6 | |
| Total: | 30.5 | uL |

It is contemplated that fractions or multiples of these values can be used in accordance with the present invention it reagent volume ratios are preserved.

PCR Thermal Cycler Program

Step 1: Initial Denaturation

| Time (min) | Temperature (° C.) |
|---|---|
| 10:00 | 95 |

Step 2: 20 Cycles of Touchdown PCR

| Time (min) | Temperature (° C.) | Touchdown |
|---|---|---|
| 0:20 | 95 | none |
| 1:00 | 63 | decrease by 0.5° C. |
| | | each cycle |
| 1:00 | 72 | none |

Touchdown or step-down PCR refers to the incremental decrease of the annealing temperature with each cycle. Here, the annealing temperature was decreased by 0.5° C. in each cycle. The objective was to increase the efficiency of each successive amplification step, while maintaining more rigorous primer specificity in the initial amplification steps.

Step 3: 25 Cycles of Regular PCR

| Time (min) | Temperature (° C.) |
|---|---|
| 0:20 | 95 |
| 0:45 | 56 |
| 1:00 | 72 |

Step 4: Final Elongation

| Time (min) | Temperature (° C.) |
|---|---|
| 7:00 | 72 |
| HOLD | 6 |

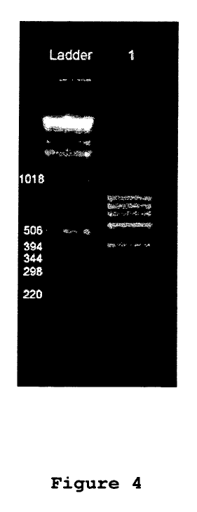

Primer pairs designed in accordance with a primer design protocol of the present invention fire herein provided as SEQ ID Nos. 7-26. Primer design, concentrations of PCR reagents and PCR reaction mixture were prepared as described above. A GeneAmp PCR System 9600 thermal cycler was programmed as detailed above. PCR products were detected by agarose gel electrophoresis, followed by staining with ethidium bromide. PCR products were identified based on size comparison to a standard DNA ladder, FIG. 4 shows the results of this experiment.

Ten genes expressed in methicillin-resistant Staphylococcus aureus (MRSA) were identified from GenBank, as outlined hereinbelow. Detection of 10 genetics targets, corresponding to these ten different genes in methicillin-resistant Staphylococcus aureus was achieved on Agarose gel, in accordance with a method of the present invention (FIG. 4). As illustrated in FIG. 4 (Lane 1), from top to bottom, the ten genes are: (1) agr, 823 bp (ACCESSION M21854) (2) clumping factor, 726 bp (ACCESSION Z18852); (3) 16S rRNA, 653 bp (ACCESSION X68417); (4) hld, 554 bp (ACCESSION X17301); (5)^{- femA,} 419 bp (ACCESSION X17688 M23918); (6) rho, 376 bp (ACCESSION AF333962) (7) DNA polymerase III, 314 bp (ACCESSION Z48003 L39156); (8) nuclease, 282 bp (ACCESSION V01281 J01785 M10924); (9) 23S rRNA, 244 bp, (ACCESSION X68425); and (10) hsp60, 212 bp (ACCESSION AF060189).

### EXAMPLE 3

Method

A 0.5 McFarland standard (1×10⁸ CFU/mL) of MRSA bacteria was made up in sterile saline. Serial dilutions were made using sterile saline. The bacteria from 100 uL of each dilution were pelleted and resuspended in Lysis Buffer (50 mM Tris-HCl (pH 8.0), 50 mM NaCl, and 5 mM EDTA (pH 8.0) for DNA extraction. Colony counts of bacteria on agar plates corroborated the accuracy of the McFarland standard. 2 uL from each DNA sample (100 uL each) was used for the method of the present invention as described herein above.

Results

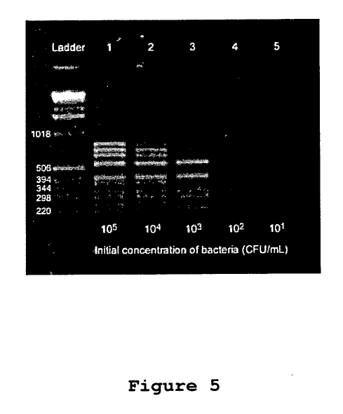

The sensitivity of the method of the present invention is illustrated in FIG. 5, with 10 amplicons visible in Lanes 1 and 2. Lanes 3 to 5 show progressive loss of amplified targets with a decrease in the initial concentration (CFU/mL) of bacteria. A theoretical detection limit of approximately 500 CFU/mL (2 uL/PCR reaction*500 CFU/1000 uL=1 CFU/PCR reaction) was indicated in this example.

### EXAMPLE 4

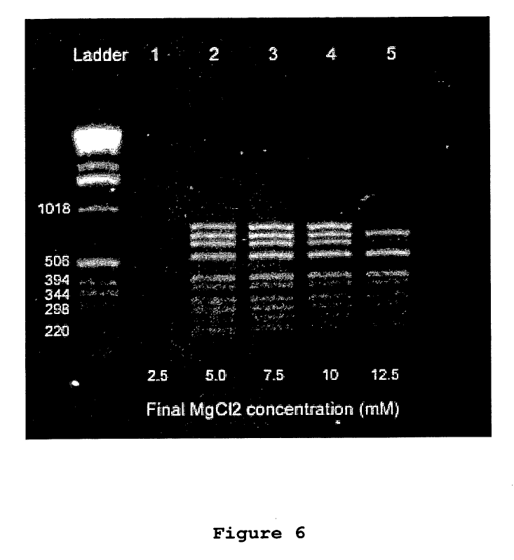

The effect of varying magnesium chloride concentrations was investigated in accordance with an embodiment of the present invention. Final MgCl₂ concentrations of 5.0 mM to 10.0 mM were shown to allow detection of 10 target genes by agarose gel electrophoresis in accordance with the method described in Example 2(FIG. 6—Lanes 2,3 &4). No amplicons were detected when a final concentration of 2.5 mM MgCl₂ was provided in the PCR reaction mixture (Lane 1). Only 9 amplicons were visible (Lane 5) when the final concentration of MgCl₂ was increased to 12.5 mM.

### EXAMPLE 5

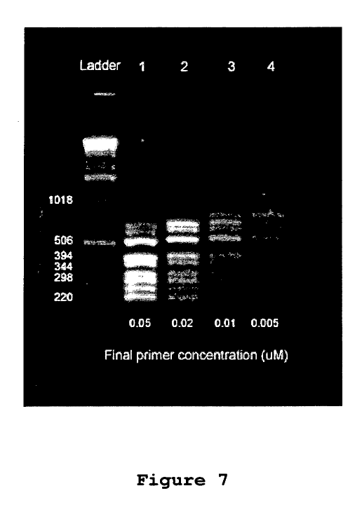

The effects of varying primer concentrations were also studied (FIG. 7). Final primer concentrations were of 0.005 μm, 0.01 μm, 0.02 μm and 0.05 μm were tested.

At final primer concentrations 0.05 μm and 0.02 μm only 9 amplicons were visible (FIG. 7, Lanes 1 and 2). 10 amplicons were visible at final primer concentrations of 0.01 μm and 0.005 μm (FIG. 7, Lanes 3 and 4).

### CONCLUSION

The present invention provides a single set of multiplex PCR conditions that will work with primers designed according to the primer design protocol as detailed herein. By employing this single set of multiplex PCR conditions, primer pairs designed in accordance with the primer design protocol of the present invention can be added or removed without having to change the reaction conditions of the enhanced multiplex PCR method.

The present invention amplifies all of the genetic targets if they are present. At least ten genetic targets can be amplified simultaneously, according to one embodiment of the present invention. More efficiently amplified primers do not overwhelm less efficiently amplified primers in the reaction mix. A particular advantage of the present invention is that amplification is efficient enough to produce PCR products or amplicons that can be detected with inexpensive and simple methods like agarose gel electrophoresis with ethidium-bromide staining.

As indicated in Examples 3, 4 and 5, a method of the present invention can detect at least 10 genetic targets. According to an embodiment of the present invention, detection can be accomplished by agarose gel electrophoresis. A starting bacterial concentration (before DNA extraction) of 1×10⁴ CFU/mL or greater is preferred. According to another embodiment of the present invention, a final MgCl2 concentration of 7.5-10 mM in the PCR reaction mixture is preferred for optimal amplification and detection of at least 10 genetic targets. A final primer concentration between 0.005 and 0.05 μM is also preferred in order to amplify at least 10 genetic targets.

In effect, the present invention creates a common platform that researchers and clinicians can use to develop new multiplex PCR tests. If a new genetic target is discovered, the method of the present invention can be adapted to include this target in the reaction mixture for amplification with the existing targets without requiring any changes to reaction conditions. Conversely, if a certain genetic target is no longer desirable, its primer pair can be removed from the existing assay, without requiring any changes to the reaction conditions.

To summarize, the present invention includes the advantages of (1)significant savings in reagent costs and technologist time and labour due to the elimination of optimization procedures; (2) rapid product development: different PCR primer pairs will work together right away without competitive inhibition; and (3) flexibility: new PCR primers can be added or subtracted from existing assays without adjusting reaction conditions.

By selecting target-specific primers, the present invention can be used to develop diagnostic assays in a variety of fields. For example, the present invention can be used to provide high-throughput, sensitive and specific diagnostic tests for infectious diseases, screening panels for genetic diseases, or for the detection of disease-causing genes. In particular, the present invention can be tailored to provide a screening method and kit to detect potential causative organisms of human encephalitis in cerebrospinal (CSF) samples (e.g. herpes simplex virus, human herpes virus 6, Cryptococcus, Listeria, H. influenzae type B, S. pneumoniae, E. coli, etc.) The present invention also finds application in the fields of blood product screening and genetic testing such as prenatal screening for genetic diseases and detection of cancer-causing genes in pathology samples, for example.

In the case of diseases such as meningitis/encephalitis, there are at least 10 different bacteria and viruses that can cause the classic symptoms of fever, stiff neck, and altered mental status. Unfortunately, it is prohibitively expensive to test for all 10 organisms individually. As a result doctors are usually forced to treat patients empirically, without adequate knowledge of the true cause of disease. Empirical treatment is expensive, promotes antibiotic resistance, and puts patients at risk of adverse drug reactions. It may not even cover the rarer causes of disease. The present invention allows for the simultaneous detection of multiple genetic targets, such as genetic disease markers of a disease of interest, thereby allowing a physician to quickly, reliably and economically arrive at an accurate diagnosis.

The present invention may be provided as a diagnostic kit. A kit of the present invention may be tailored for the diagnosis of a variety of diseases and/or conditions such as meningitis/encephalitis, STDs, respiratory infections, etc. According to one embodiment of the present invention, a kit will contain PCR reaction tubes that are pre-loaded with a PCR reaction mixture. Such a kit may also include multiple target-specific primer pairs for the amplification and detection of genetic targets of interest. According to one embodiment of the invention, each tube will be ready for immediate use, and the laboratory technologist will simply add sample DNA and then run it using standard PCR equipment. Alternatively, a kit of the present invention may include some or all of the ingredients necessary to establish the suitable reaction conditions of the invention, such as a PCR reaction mixture, enzymes, and target-specific primers for example. A kit of the present invention may include predefined measures of the components for performing multiple repeats of the enhanced multiplex PCR method. Test controls may also be provided as a component of the kit of the present invention to confirm the reaction conditions of a given amplification reaction. For example, positive and negative controls may be provided. In the case of a bacterial target, highly conserved targets like 16 s rRNA and 23 s rRNA genes may be employed. In the case of human molecular testing, human GAPDH or beta-globin genes may be used as positive test controls. These are highly conserved genes that can be used to confirm the presence of human DNA in a sample. Similarly, negative test controls may also be employed to provide indicators of the reaction conditions. A kit of the present invention preferably includes a set of instructions for practicing the method thereof. The kits of the present invention may be designed to be compatible with the major brands of PCR machines. Alternatively, the kits of the present invention may be tailored for a fully automated system specific to the present invention with integrated DNA extraction, and PCR amplification and detection functions.

The embodiment(s) of the invention described above is(are) intended to be exemplary only. The scope of the invention is therefore intended to be limited solely by the scope of the appended claims.

TABLE 1
PCR analysis of BacT/Alert blood cultures
for mecA, nuc and 16S rRNA genes

| | | No. of | mecA Result | | nuc Result | |
|---|---|---|---|---|---|---|
| Organism | | bottles | No. of | No. of | No. | No. of |
| initially | No. of | positive | mecA | mecA | of nuc | nuc |
| reported from | bottles | for 16S | positive | negative | positive | negative |
| bottles | tested | rRNA | bottles | bottles | bottles | bottles |
| ^{a}MS-S. aureus | 20 | 20 | 0 | 20 | 20 | 0 |
| ^{b}MR-S. aureus | 2 | 2 | 2 | 0 | 2 | 0 |
| MS-S. aureus | 1 | 1 | 1 | 0 | 1 | 0 |
| and MR-CoNS | | | | | | |
| MR-CoNS | 27 | 26^{c} | 25 | 1 | 0 | 26 |
| MS-CoNS | 28 | 28 | 4 | 24 | 0 | 28 |
| MR-CoNS and | 3 | 3 | 3 | 0 | 0 | 3 |
| MS-CoNS | | | | | | |
| Non- | 19 | 18′ | 0 | 18 | 0 | 18 |
| staphylococcal | | | | | | |
| isolates^{e} | | | | | | |

^{a}MS, methicillin susceptible.
^{b}MR, methicillin resistant.
^{c}One bottle was PCR negative for 16S rRNA, mecA and nuc genes, indicating PCR inhibition.
^{d}This isolate did not grow for susceptibility testing at time blood culture results was reported. Subsequent testing of the organism indicated an oxacillin MIC s 0.25 ntg/I, disk diffusion zone diameter of 25.5 nun (S), and negative PCR for mecA.
^{e}These bottles contained Enterococcus spp. (5 bottles), Streptococcus spp. (9 bottles), Coryneformbacteria (1 bottle), and Gram-negative bacilli (4 bottles).

TABLE 2
Analysis of isolates with oxacillin susceptibility results
that were discrepant with the blood culture bottle result.

| | | | Isolate retest results | | |
|---|---|---|---|---|---|
| | Isolate from this | mecA | Oxacilin | | |
| Bottle | bottle initially | PCR | susceptibility | Oxacillin | mecA |
| Number | reported as | of bottle | by DD | MIC | PCR |
| 10 | ^{a}MR-CoNS | − | S | 0.5 | − |
| 8 | ^{b}MS-CoNS | + | R | 2.0 | + |
| 92 | MS-CoNS | + | R | ND^{c} | + |
| 93 | MS-CoNS | + | R | ND | + |
| 94 | MS-CoNS | + | R | ND | + |

^{a}MR, methicillin resistant
^{b}MS, methicillin susceptible
^{c}ND: Not done.

I/we claim:

1. A method for simultaneously amplifying multiple genetic targets, said method comprising:
selecting primer pairs specific to said multiple genetic targets according to a primer selection criteria;
effecting a hot start initiation of amplification of said multiple genetic targets in a single reaction vessel together with said primer pairs and a PCR reaction mixture;
performing a series of PCR reaction steps to amplify each of said multiple genetic sequences in said sample;
wherein said PCR reaction mixture is adaptable for simultaneously amplifying multiple genetic targets in said single reaction vessel without requiring optimization of pre-set amplification reaction conditions of said method when said primer pairs are provided at a final concentration of approximately 0.005 μM-0.05 μM in the reaction mixture.

2. The method of claim 1 wherein each of said primer pairs are provided to have a final concentration of approximately 0.01 μM in said reaction mixture.

3. The method of claim 1 wherein said PCR reaction mixture includes at least 5.0 mM MgCl₂.

4. The method of claim 1 wherein said primer selection criteria includes selecting primer pairs having a melting temperature in the range of 55 to 65° C.

5. The method of claim 4 wherein said melting temperatures of said pre-selected primer pairs are within a 2° C. range of variation.

6. The method of claim 5 wherein the melting temperatures of said pre-selected primer pairs are 55-57° C.

7. The method of claim 1 wherein said primer selection criteria includes selecting primer pairs having a GC content of 40-50%.

8. The method of claim 1 wherein said primer selection criteria includes selecting primer pairs that are 18-27 nucleotides in length.

9. The method of claim 1 wherein said series of PCR reaction steps includes a first and second PCR step.

10. The method of claim 1 wherein said series of PCR reaction steps includes a step of touchdown PCR.

11. The method of claim 10 wherein said step of touchdown PCR is performed in a first set of PCR steps.

12. The method claim 10 wherein said first set of PCR steps includes 20 cycles of touchdown PCR.

13. The method of claim 9 wherein said second set of reaction steps includes at least 20 cycles of PCR.

14. The method of claim 13 wherein said second set of reaction steps includes 25 cycles of PCR.

15. A method for preparing a PCR reaction mixture for simultaneously multiplexing multiple genetic targets, said method comprising:
adjusting the final MgCl₂ concentration of a PCR buffer suitable for a PCR reaction to at least 5.0 mM; and
adding a hot start initiation means to said buffer;
wherein said PCR reaction mixture is adaptable for simultaneously multiplexing multiple genetic targets in the presence of pre-selected primer pairs having a final concentration of 0.005-0.05 μM when added to said mixture.

16. The method of claim 15 wherein each of said pre-selected primer pairs are provided to have a final concentration of approximately 0.01 μM in said reaction mixture.

17. The method of claim 15 wherein the final concentration of MgCl₂ in said PCR reaction mixture is approximately 7.5 mM.

18. The method of claim 15 wherein said pre-selected primer pairs are selected to be and 18-27 nucleotides in length; have a melting temperature in the range of 55 to 65° C.; and a GC content of 40-50%.

19. The method of claim 18 wherein said melting temperatures of said pre-selected primer pairs are within a 2° C. range of variation.

20. The method of claim 19 wherein the melting temperatures of said pre-selected primer pairs are 55-57° C.

21. A method for simultaneously amplifying multiple genetic targets, said method comprising:
mixing a sample to be tested for the presence of said multiple genetic targets with pre-selected primer pairs specific to said multiple genetic targets and a PCR reaction mixture;
effecting a hot start initiation of amplification of said genetic targets;
performing a series of PCR reaction steps to amplify each of said multiple genetic sequences in said sample;
wherein said pre-selected primer pairs are provided to optimize amplification of said multiple genetic targets in said reaction mixture in the absence of requiring optimization of reaction conditions of said method.

22. The method of claim 21 wherein each of said primer pairs are provided to have a final concentration in said reaction mixture of 0.005-0.05 μM.

23. The method of claim 22 wherein each of said primer pairs are provided to have a final concentration of approximately 0.01 μM in said reaction mixture.

24. The method of claim 21 wherein said PCR reaction mixture includes at least 5.0 mM MgCl₂.

25. The method of claim 21 wherein said PCR reaction mixture includes approximately 7.5 mM MgCl₂.

26. The method of claim 21 wherein said pre-selected primer pairs are selected to be 18-27 nucleotides in length; have a melting temperature in the range of 55 to 65° C.; and have a GC content of 40-50%.

27. The method of claim 26 wherein said melting temperatures of said pre-selected primer pairs are within a 2° C. range of variation.

28. The method of claim 27 wherein the melting temperatures of said pre-selected primer pairs are 55-57° C.

29. The method of claim 21 wherein said series of PCR reaction steps includes a first and second PCR step.

30. The method of claim 21 wherein said series of PCR reaction steps includes a step of touchdown PCR;

31. The method of claim 30 wherein said step of touchdown PCR is performed in a first set of PCR steps.

32. The method claim 29 wherein said first set of PCR steps includes 20 cycles of touchdown PCR.

33. The method of claim 29 wherein said second set of reaction steps includes at least 20 cycles of PCR.

34. The method of claim 33 wherein said second set of reaction steps includes 25 cycles of PCR.

35. A method for simultaneously detecting multiple genetic targets, said method comprising:
mixing a sample to be tested for the presence of said multiple genetic targets with pre-selected primer pairs specific to said multiple genetic targets and a PCR reaction mixture;
effecting a hot start initiation of amplification of said genetic targets;
performing a series of PCR reaction steps to amplify each of said multiple genetic sequences in said sample; and
detecting said multiple genetic targets present in said sample;
wherein said pre-selected primer pairs are provided to optimize amplification of said multiple genetic targets in said PCR reaction mixture in the absence of requiring optimization of reaction conditions of said method.

36. The method of claim 35 wherein gel electrophoresis is employed in detecting said multiple genetic targets.

37. The method of claim 35 wherein each of said primer pairs are provided to have a final concentration in said reaction mixture of 0.005-0.05 M.

38. The method of claim 35 wherein each of said primer pairs are provided to have a final concentration of approximately 0.01 μM in said reaction mixture.

39. The method of claim 35 wherein said PCR reaction mixture includes at least 5.0 mM MgCl₂.

40. The method of claim 35 wherein said PCR reaction mixture includes approximately 7.5 mM MgCl₂.

41. The method of claim 35 wherein said pre-selected primer pairs are selected to be 18-27 nucleotides in length; have a melting temperature in the range of 55 to 65° C.; and have a GC content of 40-50%.

42. The method of claim 41 wherein said melting temperatures of said pre-selected primer pairs are within a 2° C. range of variation.

43. The method of claim 42 wherein the melting temperatures of said pre-selected primer pairs are 55-57° C.

44. The method of claim 35 wherein said series of PCR reaction steps includes a first and second PCR step.

45. The method of claim 35 wherein said serials of PCR reaction steps includes a step of touchdown PCR;

46. The method of claim 45 wherein said step of touchdown PCR is performed in a first set of PCR steps.

47. The method claim 44 wherein said first set of PCR steps includes 20 cycles of touchdown PCR.

48. The method of claim 44 wherein said second set of reaction steps includes at least 20 cycles of PCR.

49. The method of claim 43 wherein said second set of reaction steps includes 25 cycles of PCR.

50. The method of claim 44 wherein said first series of PCR reaction steps comprises 20 cycles of touchdown PCR including 20 s at 95° C., 1 min at 63° C.—decreased by 0.5 each cycle, and 1 min at 72° C.

51. The method of claim 44 wherein said second series of PCR reaction steps comprises 25 cycles of PCR, including 20 s at 95° C., 45 s at 56° C. and 1 min at 72° C.

52. The method of claim 51 including 7 min at 72° C.

53. The method of claim 1 wherein said multiple genetic targets are DNA sequences.

54. The method of claim 53 wherein DNA sequences are selected from the group consisting of bacterial DNA, viral DNA, plant DNA, animal DNA or human DNA.

55. The method of claim 9 wherein said first series of PCR reaction steps comprises 20 cycles of touchdown PCR including 20 s at 95° C., 1 min at 63° C.—decreased by 0.5 each cycle, and 1 min at 72° C.

56. The method of claim 9 wherein said second series of PCR reaction steps comprises 25 cycles of PCR, including 20 s at 95° C., 45 s at 56° C. and 1 min at 72° C.

57. The method of claim 56 including 7 min at 72° C.

58. The method of claim 9 wherein said multiple genetic targets are DNA sequences.

59. The method of claim 58 wherein DNA sequences are selected from the group consisting of bacterial DNA, viral DNA, plant DNA, animal DNA or human DNA.

60. The method of claim 29 wherein said first series of PCR reaction steps comprises 20 cycles of touchdown PCR including 20 s at 95° C., 1 min at 63° C.—decreased by 0.5 each cycle, and 1 min at 72° C.

61. The method of claim 29 wherein said second series of PCR reaction steps comprises 25 cycles of PCR, including 20 s at 95° C., 45 s at 56° C. and 1 min at 72° C.

62. The method of claim 61 including 7 min at 72° C.

63. The method of claim 29 wherein said multiple genetic targets are DNA sequences.

64. The method of claim 63 wherein DNA sequences are selected from the group consisting of bacterial DNA, viral DNA, plant DNA, animal DNA or human DNA.

65. A PCR reaction mixture for use in simultaneously amplifying multiple genetic targets, said mixture comprising:
a PCR buffer reagent including 5 mM-10 mM MgCl₂;
wherein said PCR reaction mixture is adaptable for simultaneously amplifying multiple genetic targets in a single reaction vessel without requiring optimization of pre-set amplification reaction conditions.

66. A PCR reaction mixture for use in simultaneously amplifying multiple genetic targets, said mixture comprising:
a PCR buffer reagent including 5 mM-10 mM MgCl₂;
wherein said PCR reaction mixture is adaptable for simultaneously amplifying multiple genetic targets in a single reaction vessel without requiring optimization of pre-set amplification reaction conditions.

67. A PCR reaction mixture for use in simultaneously amplifying multiple genetic targets in an amplification reaction, said mixture comprising:
a PCR buffer reagent including 5 mM-10 mM MgCl₂;
dNTPs having a final concentration of approximately 0.25 mM each; and
pre-selected primer pairs corresponding to target genetic sequences to be amplified; each of said primers having a final concentration of approximately 0.005 μM-0.05 82 M in the reaction mixture;
wherein said PCR reaction mixture is adaptable for simultaneously amplifying multiple genetic targets in a single reaction vessel without requiring optimization of pre-set amplification reaction conditions.

68. A kit for simultaneously amplifying multiple genetic targets for detection, said kit comprising:
a PCR reaction mixture having a final MgCl₂ concentration of 5-12.5 mM;
a set of pre-selected, target-specific primer pairs corresponding to each of said multiple genetic targets; and
a set of instructions for using contents of said kit for simultaneously amplifying multiple genetic targets in a sample to be tested;
wherein said PCR reaction mixture is adaptable for simultaneously amplifying multiple genetic targets in a single reaction vessel without requiring optimization of pre-set amplification reaction conditions when said pre-selected, target-specific primer pairs are provided at a final concentration of approximately 0.005 μM -0.05 μM in the reaction mixture.

69. The kit of claim 68 wherein said PCR reaction mixture is pre-loaded in at least one reaction vessel.

70. The kit of claim 69 wherein said at least one reaction vessel further includes said set of pre-selected, target-specific primer pairs.

71. The kit of claim 68 further comprising, a DNA polymerase enzyme.

72. The kit of claim 71 wherein said DNA polymerase enzyme is a hot start enzyme.

73. A method for simultaneously amplifying multiple genetic targets, said method comprising:
mixing a sample to be tested for the presence of said multiple genetic targets with pre-selected primer pairs specific to said multiple genetic targets and a PCR reaction mixture including a final MgCl₂ concentration of at least 5.0 mM;
effecting means for a hot start initiation of amplification of said genetic targets; and
performing a series of PCR reaction steps including a step of touchdown PCR;
wherein said amplification is optimized when said pre-selected primer pairs are provided to have a final concentration of 0.005-005 μM in said reaction mixture.

74. The method of claim 73 wherein each of said pre-selected primer pairs are provided to have a final concentration of approximately 0.01 μM.

75. The method of claim 73 wherein said pre-selected primer pairs are selected to have a melting temperature in the range of 55 to 65° C.

76. The method of claim 75 wherein said melting temperatures of said pre-selected primer pairs are within a 2° C. range of variation.

77. The method of claim 76 wherein the melting temperatures of said pre-selected primer pairs are 55-57° C.

78. The method of claim 73 wherein each of said pre-selected primer pairs include a GC content of 40-50%.

79. The method of claim 73 wherein each of said pre-selected primer pairs is 18-27 nucleotides in length.

80. The method of claim 79 wherein each of said pre-selected primer pairs is 22 nucleotides in length.

81. The method of claim 73 wherein the final concentration of MgCl₂ in said PCR reaction mixture is 5 to 12.5 mM.

82. The method of claim 81 wherein said PCR reaction mixture includes more than 6 mM of MgCl₂.

83. The method of claim 82 wherein said PCR reaction mixture includes 7.5 mM of MgCl₂.

84. The method of claim 73 wherein ten or more genetic targets are simultaneously detected.

85. The method of claim 73 wherein said means for effecting a hot start initiation is a hot start enzyme.

86. The method of claim 85 wherein said hot start enzyme is a Taq Polymerase enzyme.

87. The method of claim 86 wherein said enzyme is Amplitaq Gold™.

88. The method of claim 73 wherein said means for effecting a hot start initiation includes a DNA polymerase enzyme and a heating step.

89. A method for simultaneously detecting multiple genetic targets in a sample to be tested, said method comprising:
selecting primer pairs corresponding to said multiple genetic targets according to a pre-defined primer selection criterion;
mixing said primer pairs with said sample to be tested and a PCR reaction mixtures; said PCR reaction mixture including a final concentration of at least 5.0 mM MgCl₂;
effecting means for a hot start initiation of amplification of said genetic targets;
performing a series of PCR reaction steps including a step of touchdown PCR; and
detecting for the presence of said multiple genetic targets in said sample;
wherein when said primer pairs are provided in said reaction mixture to have a final concentration of 0.005-0.05 μM amplification of said multiple genetic targets is optimized in the absence of requiring optimization of reaction conditions of said method.

90. The method of claim 89 wherein each of said pre-selected primer pairs are provided to have a final concentration of approximately 0.01 μM.

91. The method of claim 89 wherein said pre-selected primer pairs are selected to have a melting temperature in the range of 55 to 65° C.

92. The method of claim 91 wherein said melting temperatures of said pre-selected primer pairs are within a 2° C. range of variation.

93. The method of claim 92 wherein the melting temperatures of said pre-selected primer pairs are 55-57° C.

94. The method of claim 89 wherein each of said pre-selected primer pairs include a GC content of 40-50%.

95. The method of claim 89 wherein each of said pre-selected primer pairs is 18-27 nucleotides in length.

96. The method of claim 95 wherein each of said pre-selected primer pairs is 22 nucleotides in length.

97. The method of claim 89 wherein said PCR reaction mixture includes 5 to 12.5 mM of MgCl₂.

98. The method of claim 97 wherein said PCR reaction mixture includes 5 to 10 mM of MgCl₂.

99. The method of claim 98 wherein said PCR reaction mixture includes more than 6 mM of MgCl₂.

100. The method of claim 99 wherein said PCR reaction mixture includes 7.5 mM of MgCl₂.

101. The method of claim 89 wherein ten or more genetic targets are simultaneously detected.

102. The method of claim 89 wherein said means for effecting a hot start initiation is a hot start enzyme.

103. The method of claim 102 wherein said hot start enzyme is a Taq Polymerase enzyme.

104. The method of claim 103 wherein said enzyme is Amplitaq Gold™.

105. The method of claim 89 wherein said means for effecting a hot start initiation includes a DNA polymerase enzyme and a heating step.

106. The method of claim 89 wherein said series of PCR reaction steps includes 20 cycles of touchdown PCR including 20 s at 95° C., 1 min at 63° C.—decreased by 0.5 each cycle, and 1 min at 72° C.

107. The method of claim 106 wherein said series of PCR reaction steps further includes 25 cycles of PCR, including 20 s at 95° C., 45 s at 56° C. and 1 min at 72° C.

108. The method of claim 107 further including 7 min at 72° C.

109. The method of claim 89 wherein said genetic targets are DNA sequences.

110. The method of claim 109 wherein DNA sequences are selected from the group consisting of bacterial DNA, viral DNA, plant DNA, animal DNA or human DNA.

111. The method of claim 73 wherein said series of PCR reaction steps includes 20 cycles of touchdown PCR including 20 s at 95° C., 1 min at 63° C.—decreased by 0.5 each cycle, and 1 min at 72° C.

112. The method of claim 111 wherein said series of PCR reaction steps further includes 25 cycles of PCR, including 20 s at 95° C., 45 s at 56° C. and 1 min at 72° C.

113. The method of claim 112 further including 7 min at 72° C.

114. The method of claim 73 wherein said genetic targets are DNA sequences.

115. The method of claim 114 wherein DNA sequences are selected from the group consisting of bacterial DNA, viral DNA, plant DNA, animal DNA or human DNA.
